(19) United States
(12) Patent Application Publication
(10) Pub. No.: US 2023/0190935 A1
(43) Pub. Date: Jun. 22, 2023

(54) Blood pathogen inactivation method

(71) Applicant: Institute of Blood Transfusion CAMS, Chengdu (CN)

(72) Inventors: Zhong LIU, Chengdu (CN); Yundi YIN, Chengdu (CN); Ling LI, Chengdu (CN); Haixia XU, Chengdu (CN)

(73) Assignee: Institute of Blood Transfusion CAMS, Chengdu (CN)

(21) Appl. No.: 18/085,597

(22) Filed: Dec. 21, 2022

(30) Foreign Application Priority Data
Dec. 22, 2021 (CN) 202111584844 .1

(51) Int. Cl. A61K 41/17

(52) U.S. Cl. CPC A61K 41/17; A61L 2202/22

(57) ABSTRACT

The present disclosure provides a pathogen inactivation method, which is low-frequency sonication together with illumination of a photosensitizer-containing blood sample; and the low-frequency sonication is conducted at a frequency of 15-500 KHz. Through the combination of sonication and photochemical pathogen inactivation technology that enhance and complement each other, the blood pathogen inactivation method provided by the present disclosure enhances a pathogen inactivation effect, reduces a dosage of the photosensitizer, photosensitizer-related blood quality damage, energy demand for the illumination, and pathogen inactivation treatment time, increases the blood illumination thickness for effective pathogen inactivation, saves illumination bag materials, shortens the size of illumination equipment, saves costs, and helps the pathogen inactivation technology go to the market.

### CROSS REFERENCE TO RELATED APPLICATION

This patent application claims the benefit and priority of Chinese Patent Application No. 202111584844.1, filed with the China National Intellectual Property Administration on Dec. 22, 2021, the disclosure of which is incorporated by reference herein in its entirety as part of the present application.

### TECHNICAL FIELD

The present disclosure specifically relates to a blood pathogen inactivation method.

### BACKGROUND

Blood transfusion is a routine clinical treatment method and first aid method. The pathogens that have not been detected by routine testing procedures and are carried by the blood can be transmitted to patients, which bring immeasurable harm to the health of patients. Blood safety is related to people’s health and national security. The current methods for controlling transfusion-transmitted pathogens include blood donor screening before donation and blood screening, which substantially reduce the risk of transfusion-transmitted pathogens. However, the current blood pathogen detection methods including nucleic acid testing (NAT) has problems such as a “window period” and limited types of routinely screened pathogens. There has been a risk of blood-transmitted pathogens. Therefore, there is an urgent need to develop a new technique to reduce the risk of transfusion-transmitted pathogens.

The pathogens that may be carried in the blood include Gram-positive cocci: Staphylococcus aureus, coagulase-negative staphylococci, Streptococcus pneumoniae, Streptococcus viridans, and Enterococcus sp.; Gram-positive bacilli: Mycobacterium tuberculosis and Listeria monocytogenes; Gram-negative cocci: Neisseria meningitidis and Branhamella catarrhalis; Gram-negative bacilli: Escherichia coli, Pseudomonas aeruginosa, Klebsiella sp., Serratia sp., Salmonella sp., Acinetobacter sp., Legionella pneumophila, and Haemophilus sp.; fungi: Candida sp., Aspergillus sp., Cryptococcus sp., and Coccidioides sp.; anaerobes: Bacteroides sp. and Clostridium perfringens; viruses (hepatitis A virus, hepatitis B virus, hepatitis C virus, human immunodeficiency virus, etc.); parasites (malaria, etc.); fungi; prions, etc.

Pathogen inactivation technology can effectively solve the “window period” problem existing in the blood pathogen detection technology, reduce or eliminate the risk of transfusion-transmitted pathogens, and lower the risk of blood transfusion infection of pathogens and emerging pathogens from unconventional screening. In addition, the pathogen inactivation technology can inactivate leukocytes, and reduce the incidence of graft-versus-host disease.

Pathogen inactivation technologies currently under study include photochemical pathogen inactivation technology (methylene blue photochemical method, psoralen photochemical method, and riboflavin photochemical method), ultraviolet-C irradiation, low-temperature plasma technology, femtosecond laser/ultrashort pulse laser technology, high hydrostatic pressure technology, etc. The technologies approved for use outside of China include methylene blue photochemical method, psoralen photochemical method, and riboflavin photochemical method. The method approved for the inactivation of blood pathogens on the market in China is mainly the methylene blue photochemical method. Due to the residual toxicity of methylene blue, methylene blue use is restricted or prohibited in Europe and the United States. Therefore, it is necessary for China to develop novel blood pathogen inactivation technologies for blood stations or clinical use to reduce the risk of transfusion-transmitted pathogens.

In 1980, ultrasonic inactivation of pathogens was first proposed in food science as an “emerging technology” (FDA, 2000), and then it was studied in other fields for similar purposes. After 1990, it was found that sonication could inactivate target microorganisms, and significant progress was made in the inactivation effect of sonication on microorganisms. The inactivation effect of sonication alone on pathogens cannot meet the food and drug safety standards, and methods for better pathogen inactivation should be explored, but at present, there is no research on the technology that combines sonication with antimicrobial agents, chemicals, heat, or pressure.

### SUMMARY

To solve the above problem, the present disclosure provides a blood pathogen inactivation method, including the following steps: drawing blood, adding a photosensitizer, and conducting illumination and low-frequency sonication simultaneously, where low-frequency sonication is conducted at a frequency of 15-500 KHz.

Further, the photosensitizer may be riboflavin.

Further, a concentration of the riboflavin in a blood sample may be 10-100 µmol/L.

Still further, the concentration of the riboflavin in the blood sample may be 50 µmol/L.

Still further, the riboflavin may be dissolved in normal saline and added with the blood sample.

Still further, a content of the riboflavin in the normal saline may be 50-500 µmol/L.

Still further, the content of the riboflavin in the normal saline may be 500 µmol/L.

Further, the low-frequency sonication and the illumination may last for 3 min to 2 h.

Still further, the low-frequency sonication and the illumination may last for 10-30 min.

Still further, the low-frequency sonication may be conducted at a frequency of 30 KHz.

Still further, the illumination may be conducted at a wavelength of 311 ± 50 nm.

Further, the blood sample may be one selected from the group consisting of plasma, platelets, suspended red blood cells, and whole blood.

Through the combination of sonication and photochemical pathogen inactivation technology that enhance and complement each other, the blood pathogen inactivation method provided by the present disclosure enhances a pathogen inactivation effect, reduces a dosage of the photosensitizer, photosensitizer-related blood quality damage, energy demand for the illumination, and pathogen inactivation treatment time, increases the blood illumination thickness for effective pathogen inactivation, saves illumination bag materials, shortens the size of illumination equipment, saves costs, and helps the pathogen inactivation technology go to the market.

Obviously, according to the above-mentioned content of the present disclosure, other various forms of modification, substitution or change can also be made based on the common technical knowledge and conventional means in the art without departing from the above-mentioned basic technical idea of the present disclosure.

The above-mentioned content of the present disclosure will be further described in detail below through the specific implementation in the form of examples. However, they should not be construed as limiting the scope of the above-mentioned subject of the present disclosure to the following examples. All technologies implemented based on the above-mentioned content of the present disclosure fall within the scope of the present disclosure.

### BRIEF DESCRIPTION OF THE DRAWINGS

FIG. 1 illustrates the growth of S. aureus after treatment with different combinations of sonication, ultraviolet (UV) light and riboflavin (in log, n = 6).

### DETAILED DESCRIPTION OF THE EMBODIMENTS

### Example 1. Blood Pathogen Inactivation Method Provided by the Present Disclosure

Step 1, whole blood was taken, and a 500 µmol/L riboflavin normal saline solution was added to make the concentration of riboflavin in the whole blood be 50 µmol/L; and

Step 2, the whole blood containing riboflavin in step 1 was placed in an illumination environment at a wavelength of 311 ± 50 nm, and simultaneously sonicated at 15 KHz for 30 min to obtain pathogen-inactivated whole blood.

### Example 2. Blood Pathogen Inactivation Method Provided by the Present Disclosure

Step 1, plasma was taken, and a 200 µmol/L riboflavin normal saline solution was added to make the concentration of riboflavin in the plasma be 50 µmol/L; and

Step 2, the plasma containing riboflavin in step 1 was placed in an illumination environment at a wavelength of 311 ± 50 nm, and simultaneously sonicated at 30 KHz for 20 min to obtain pathogen-inactivated plasma.

### Example 3. Blood Pathogen Inactivation Method Provided by the Present Disclosure

Step 1, platelets were taken, and a 100 µmol/L riboflavin normal saline solution was added to make the concentration of riboflavin in the platelets be 25 µmol/L; and

Step 2, the platelets containing riboflavin in step 1 were placed in an illumination environment at a wavelength of 311 ± 50 nm, and simultaneously sonicated at 50 KHz for 10 min to obtain pathogen-inactivated platelets.

### Example 4. Blood Pathogen Inactivation Method Provided by the Present Disclosure

Step 1, suspended red blood cells were taken, a 50 µmol/L riboflavin normal saline solution was added to make the concentration of riboflavin in the suspended red blood cells be 25 µmol/L; and

Step 2, the suspended red blood cells containing riboflavin in step 1 were placed in an illumination environment at a wavelength of 311 ± 50 nm, and simultaneously sonicated at 30 KHz for 3 min to obtain pathogen-inactivated suspended red blood cells.

### Example 5. Blood Pathogen Inactivation Method Provided by the Present Disclosure

Step 1, whole blood was taken, and a 500 µmol/L riboflavin normal saline solution was added to make the concentration of riboflavin in the whole blood be 50 µmol/L; and

Step 2, the whole blood containing riboflavin in step 1 was placed in an illumination environment at a wavelength of 311 ± 50 nm, and simultaneously sonicated at 30 KHz for 30 min to obtain pathogen-inactivated whole blood.

The beneficial effects of the present disclosure will be described below through the test example.

Test Example 1 Evaluation of the optimal combination of low-frequency sonication and photochemical method

### 1. Methods

1.1 Two bags of ABO hemolytic fresh frozen plasma (approximately 400 mL in total) were thawed and mixed well.

1.2 No more than 10% of the indicator pathogen (S. aureus or VSV) was added, and the final concentration of the pathogen was approximately 5-6log.

1.3 The samples were equally divided into two aliquots, one was added with normal saline (10% of the final sample), and the other one was added with 500 µmol/L riboflavin normal saline solution (10% of the final sample), where the concentration was 50 µmol/L after adding plasma.

1.4 The two aliquots of samples were dispensed into sterile culture plates with a diameter of 3.5 cm, 3 mL per well, and 6 wells per plate; each sample contained 9 plates finally, and there were a total of 18 plates.

1.5 The samples were divided into three groups to treat for different illumination time (with energy); each group contained 6 plates of samples (3 plates of riboflavin-containing samples, and 3 plates of riboflavin-free samples), and the treatment conditions of each group of 6 plates were as follows:
a. riboflavin-containing sample + illumination + low-frequency sonication;
b. riboflavin-containing sample + illumination;
c. riboflavin-containing sample + low-frequency sonication;
d. riboflavin-free sample + illumination + low-frequency sonication;
e. riboflavin-free sample + illumination; and
f. riboflavin-free sample + low-frequency sonication;
   the treatments for different illumination time (with energy) were as follows: the light wavelength was 311 ± 50 nm, and the illumination time (energy) was 10, 20, and 30 min (light energy was 0.27, 0.54, and 0.81 J/mL, respectively) for the three groups, respectively;
   the frequency of the low-frequency sonication was 30 KHz; in the three groups, the sonication time was synchronized with the illumination time, which was 10, 20, and 30 min, respectively; and
   the controls were the riboflavin-containing sample without illumination and the riboflavin-free sample without illumination, and the number of replicates for each group of data was N = 6.

1.6 After inactivation treatment, samples were taken, diluted 1:10, and cultured, and the pathogen growth concentration was calculated according to the Reed-Muench method.

1.7 The pathogen inactivation effect of the optimal combination of low-frequency sonication and photochemical method was analyzed.

### 2. Results

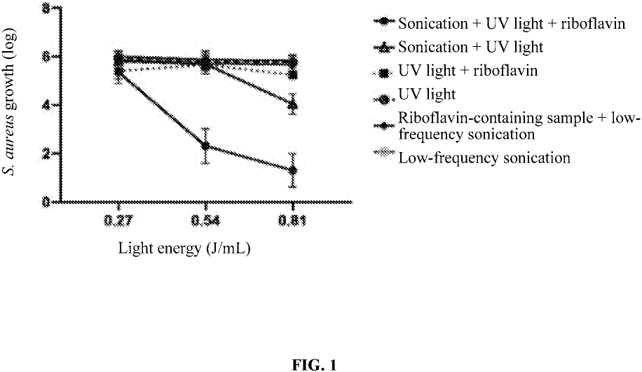

The detailed results are shown in Table 1 and FIG. 1.

Table 1 illustrates the growth of S. aureus after treatment with different combinations of sonication, UV light and riboflavin (in log, n = 6)

| Light energy (J/mL) | Sonication + UV light + riboflavin | Sonication + UV light | UV light + riboflavin | UV light | Riboflavin-containing sample + low-frequency sonication | Low-frequency sonication | Control |
|---|---|---|---|---|---|---|---|
| 0.27 | 4.48 ± 0.46 | 5.80 ± 0.17 | 5.41 ± 0.029 | 5.95 ± 0.18 | 5.84 ± 0.18 | 6.00 ± 0.19 | 5.87 ± 0.32 |
| 0.54 | 2.04 ± 0.71 | 5.69 ± 0.38 | 5.66 ± 0.09 | 5.80 ± 0.31 | 5.72 ± 0.30 | 5.87 ± 0.27 | 5.87 ± 0.32 |
| 0.81 | 1.28 ± 0.66 | 4.04 ± 0.53 | 5.23 ± 0.16 | 5.76 ± 0.06 | 5.67 ± 0.32 | 5.93 ± 0.17 | 5.87 ± 0.32 |

According to the results: the sonication + UV light + riboflavin group has a strong pathogen inactivation effect; when the light intensity reaches 0.81 J/mL, the growth of S. aureus is almost close to the detection limit level, 1.28 ± 0.66log (due to the limitation of the determination method, when the bacterial concentration of the sample is lower than 0.5log, the undetermined pathogen concentration of the sample is marked as 0.5log), while the sonication + UV light group has a poor inactivation effect when the light energy reaches 0.81 J/mL; when the light intensity reaches 0.81 J/mL, the UV light + riboflavin group, the sonication + riboflavin group, and the UV light group have no inactivation effect. It is shown that sonication can enhance the pathogen inactivation effect of the UV light, and substantially enhance the photochemical pathogen inactivation effect of UV light and riboflavin. Therefore, in the present disclosure, the combination of low-frequency sonication and riboflavin photochemical method has a synergistic effect.

In conclusion, through the combination of sonication and photochemical pathogen inactivation technology that enhance and complement each other, the blood pathogen inactivation method provided by the present disclosure enhances the pathogen inactivation effect, saves the cost of pathogen inactivation, and has practical popularization and application value.

1. A blood pathogen inactivation method, comprising the following steps:
drawing blood, adding a photosensitizer, and conducting illumination and low-frequency sonication simultaneously, wherein the low-frequency sonication is conducted at a frequency of 15-500 KHz.

2. The blood pathogen inactivation method according to claim 1, wherein the photosensitizer is riboflavin.

3. The blood pathogen inactivation method according to claim 2, wherein a concentration of the riboflavin in a blood sample is 10-100 µmol/L, and preferably 50 µmol/L.

4. The blood pathogen inactivation method according to claim 3, wherein the riboflavin is dissolved in normal saline and added with the blood sample.

5. The blood pathogen inactivation method according to claim 4, wherein a content of the riboflavin in the normal saline is 50-500 µmol/L, and preferably 500 pmol/L.

6. The blood pathogen inactivation method according to claim 1, wherein the low-frequency sonication and the illumination last for 3 min to 2 h.

7. The blood pathogen inactivation method according to claim 6, wherein the low-frequency sonication and the illumination last for 10-30 min.

8. The blood pathogen inactivation method according to claim 1, wherein the low-frequency sonication is conducted at frequency of 30 KHz.

9. The blood pathogen inactivation method according to claim 6, wherein the low-frequency sonication is conducted at frequency of 30 KHz.

10. The blood pathogen inactivation method according to claim 7, wherein the low-frequency sonication is conducted at frequency of 30 KHz.

11. The blood pathogen inactivation method according to claim 1, wherein the illumination is conducted at a wavelength of 311 ± 50 nm.

12. The blood pathogen inactivation method according to claim 6, wherein the illumination is conducted at a wavelength of 311 ± 50 nm.

13. The blood pathogen inactivation method according to claim 7, wherein the illumination is conducted at a wavelength of 311 ± 50 nm.

14. The blood pathogen inactivation method according to claim 1, wherein the blood sample is one selected from the group consisting of plasma, platelets, suspended red blood cells, and whole blood.
